(19) United States
(12) Patent Application Publication
(10) Pub. No.: US 2009/0163570 A1
(43) Pub. Date: Jun. 25, 2009

(54) BLADDER CANCER TREATMENT AND METHODS

(76) Inventors: Dorla Mirejovsky, Irvine, CA (US); Guru Reddy, Irvine, CA (US); Luigi Lenaz, Newton, PA (US)

Correspondence Address: K&L Gates LLP, 1900 MAIN STREET, SUITE 600, IRVINE, CA, 92614-7319, US

(21) Appl. No.: 12/396,158

(22) Filed: Mar. 2, 2009

Related U.S. Application Data
(63) Continuation of application No. 11/673,537, filed on Feb. 9, 2007.
(60) Provisional application No. 60/771,678, filed on Feb. 9, 2006.

(51) Int. Cl. A61K 31/404

(52) U.S. Cl. 514/414

(57) ABSTRACT

Disclosed herein are various bladder cancer treatments and methods. The present disclosure can take advantage of propylene glycol concentrations and/or NAD(P)H:quinone oxidoreductase-1 (NQO1), Cytochrome P450 Oxidoreductase (P450R) and Glucose transporter 1 (Glut-1) protein expression in human transitional cell carcinoma of the bladder to offer individually targeted bladder cancer treatments.

### CROSS-REFERENCE TO RELATED APPLICATIONS

This application is a continuation of U.S. patent application Ser. No. 11/683,537, filed Feb. 9, 2007, which claims the benefit of U.S. provisional patent application No. 60/771,678 filed Feb. 9, 2006.

### FIELD OF THE INVENTION

The present invention relates to the treatment of bladder cancer using EO9 formulations and methods. The present invention can take advantage of propylene glycol concentrations and/or NAD(P)H:quinone oxidoreductase-1 (NQO1), Cytochrome P450 Oxidoreductase (P450R) and Glucose transporter 1 (Glut-1) protein expression in human transitional cell carcinoma of the bladder to offer individually targeted bladder cancer treatments.

### BACKGROUND OF THE INVENTION

Bladder cancer is the seventh most common cancer worldwide. In 2000, it was the fourth most common cancer in men in the United Kingdom with 9,000 new cases diagnosed that year (1). In 2002, there were an estimated 280,000 cases of bladder cancer in Europe and more than 60,000 new cases were expected in the United States in 2004.

The most common type of bladder cancer (about 90%) is transitional cell carcinoma (TCC) which derives from the urothelium, the cellular lining of the urethral system (ureters, bladder and urethra). Transitional cell carcinoma (TCC) can be classified as either superficial (pTa and pT1) or muscle invasive (>pT2). Treatment of superficial TCC is currently transurethral resection (TURBT; i.e. surgical removal of all visible lesions) followed by adjuvant chemotherapy or immunotherapy. The validity of such a treatment is supported by the significant reduction in superficial tumor recurrence observed following adjuvant chemotherapy, when compared to TURBT alone (2). Whilst agents such as Mitomycin C (MMC), Epirubicin and BCG are routinely used, it is widely acknowledged that there is a need to develop either more potent and/or less toxic agents against TCC or to use current therapeutics better in terms of targeting treatment to individuals (or pathological subgroups) that are likely to benefit.

Mitomycin C (MMC) is a naturally occurring quinone based anti-neoplastic agent that belongs to a class of compounds known as bioreductive drugs (3). In general, bioreductive drugs are pro-drugs that require metabolic activation to generate cytotoxic metabolites and are all designed in principle to eradicate hypoxic cells that reside in poorly perfused regions of solid tumors. These drugs, however, can also target aerobic portions of tumors.

The key parameters that determine the cytotoxic selectivity of quinone based bioreductive drugs (i.e. between hypoxic and aerobic tumor cells) are the presence of particular enzymatic reductases required to reduce the pro-drug and the ability of molecular oxygen to reverse the activation process (4, 5) (although the relative role of reductases and oxygen tension in determining cell kill varies depending on the compound in question (4, 6)). The fact that MMC is routinely used in the treatment of TCC suggests that this disease not only possesses the appropriate biochemical machinery required for bioreductive activation but that other compounds in this class may also be useful in the treatment of this disease. Two examples of additional compounds that may also be useful include the indolequinone derivative EO9 and the aziridinyl benzoquinone RH1 (7, 8).

As stated, the ability of quinone based bioreductive drugs to eradicate aerobic or hypoxic cells is largely determined by a complex relationship between tumor enzymology including the presence of reductases and hypoxia. Several reductases have been implicated in the activation of bioreductive drugs (4, 6) although considerable attention has been paid to the enzymes Cytochrome P450 reductase (P450R) and NAD(P)H:Quinone oxidoreductase-1 (NQO1). With regards to measurement of hypoxia, endogenous markers such as Glucose transporter 1 (Glut-1) or carbonic anhydrase IX (CAIX) have been shown to correlate with exogenous hypoxia markers such as pimonidazole (9, 10). Thus, the relationship between tumor hypoxia and the expression of two key reductases in superficial and invasive transitional cell carcinomas (TCC) of the bladder is of key importance. Furthermore, the use of bladder cancer treating pharmaceutical preparations with varying penetration profiles is needed to target superficial versus muscle invasive tumors. The present invention addresses these aspects of bladder cancer treatments.

### SUMMARY OF THE INVENTION

Significant differences in NQO1 expression were found between superficial and invasive tumors with low levels observed in muscle invasive tumors. In contrast, P450R and Glut-1 were expressed in all stages and grades of TCC although expression increased with tumor stage (particularly in the case of Glut-1). In addition, Glut-1 expression was significantly elevated in G3 tumors whereas low levels of NQO1 existed. These results demonstrated that marked differences in the expression of NQO1 and Glut-1 exist between superficial and invasive bladder TCC. In addition, pharmaceutical preparations of quinone based bioreductive drugs with differing penetration profiles were found.

These results have therapeutic implications for quinone based bioreductive drugs in that single agent therapy would be appropriate for superficial disease whereas for muscle invasive disease, combination therapy using quinones to target the hypoxic fraction and other modalities to eradicate the aerobic fraction would be desirable. Furthermore, pharmaceutical preparations with lower penetration profiles can be adopted when treating superficial bladder cancers while pharmaceutical preparations with higher penetration profiles can be adopted when treating more muscle invasive bladder cancers. Taken together, these aspects of the present invention provide important advancements in the treatment of bladder cancer by allowing the tailoring of cancer treatments to the particular characteristics of an individual's disease profile.

Specifically, one embodiment according to the present invention includes a method of treating bladder cancer comprising determining the levels of at least one enzyme within a tumor and choosing a treatment based on the at least one enzyme level wherein the treatment comprises the administration of a quinone based bioreductive drug either alone or in combination with another treatment.

In another embodiment, the enzyme is selected from the group consisting of NAD(P)H:Quinone oxidoreductase-1 (NQO1) and NADPH cytochrome P450 reductase (P450R). In a particular embodiment, the enzyme is NQO1 and the treatment comprises the administration of a quinone based bioreductive drug alone. In another particular embodiment, the enzyme is NQO1 and the treatment comprises the administration of a quinone based bioreductive drug in combination with another treatment. In another particular the enzyme is P450R and the treatment comprises the administration of a quinone based bioreductive drug alone. In yet another particular the enzyme is P450R and the treatment comprises the administration of a quinone based bioreductive drug in combination with another treatment. In a further embodiment according to the present invention, the enzyme is NQO1 and P450R and the treatment comprises the administration of a quinone based bioreductive drug alone. In yet another embodiment, the enzyme is NQO1 and P450R and the treatment comprises the administration of a quinone based bioreductive drug in combination with another treatment.

One embodiment according to the present invention further comprises determining the levels of hypoxia within a tumor and choosing a treatment based on the at least one enzyme level and the hypoxia level. In a specific embodiment, the hypoxia level is determined by measuring glucose transporter 1 (Glut-1) and/or carbonic anhydrase IX (CAIX).

A particular embodiment according to the present invention includes a method of treating bladder cancer comprising choosing a treatment based on a measure selected from the group consisting of levels of NAD(P)H:Quinone oxidoreductase-1 (NQO1), levels of NADPH cytochrome P450 reductase (P450R), and levels of Glucose transporter-1 (Glut-1) wherein the treatment comprises the administration of a quinone based bioreductive drug either alone or in combination with another treatment. In various aspects of this particular embodiment: the measure can be NQO1 or P450R and the treatment comprises the administration of a quinone based bioreductive drug alone; the measure can be NQO1 or P450R and the treatment comprises the administration of a quinone based bioreductive drug in combination with another treatment; the measure can be NQO1 and P450R and the treatment comprises the administration of a quinone based bioreductive drug alone; the measure can be NQO1 and P450R and the treatment comprises the administration of a quinone based bioreductive drug in combination with another treatment; or the measure can be NQO1, P450R and Glut-1 and the treatment comprises the administration of a quinone based bioreductive drug alone or in combination with another treatment.

In one embodiment according to the present invention, the invention includes a method of treating invasive bladder cancer comprising determining the levels of NQO1 and Glut-1 within a tumor; selecting a combination treatment including a quinone based bioreductive drug in combination with another treatment based because said NQO1 level is lower and said Glut-1 level is higher than would be observed if said tumor was superficial.

In another embodiment according to the present invention, the invention includes a method of stratifying a patient for appropriate therapy for bladder cancer based on expression levels of NQ01 and Glut-1 within said patient's bladder tumor comprising: determining expression levels of NQ01 and Glut-1 within said patient's bladder tumor; and administrating a bioreductive drug as single agent therapy if said patient has superficial bladder cancer with high levels of NQ01 or administrating a combination therapy where a bioreductive drug is used in combination with radiation therapy or another chemotherapeutic agent if said patient has invasive bladder cancer with low NQ01 and high Glut-1 levels.

In particular embodiments according to the present invention, the another treatment is radiotherapy and/or the administration of at least one chemotherapeutic agent.

In various embodiments, particularly useful quinone based bioreductive drug will be selected from the group consisting of mitomycin C, the indolequinone derivative EO9, aziridinyl benzoquinone (RH1), and combinations thereof.

The present invention also includes pharmaceutical preparations. Specifically, one embodiment according to the present invention includes a pharmaceutical preparation comprising EO9 in a solution with a propylene glycol (PG) concentration selected from the group consisting of about 30% vol/vol PG, about 20% vol/vol PG, and about 10% vol/vol PG. EO9 concentrations can be present in a range from about 300 μM to about 400 μM. In a specific embodiment, the preparation comprises a solution with about a 347 μM EO9 concentration.

Pharmaceutical preparations according to the present invention can further comprise NaHCO₃, EDTA, mannitol and water. In one embodiment, the preparation comprises from about 10 mg/mL to about 120 mg/mL NaHCO₃. In a specific embodiment, the preparation comprises about 100 mg/mL or about 100.25 mg/mL NaHCO₃. In another specific embodiment the preparation comprises about 50 mg/mL NaHCO₃ or about 50.125 mg/mL NaHCO₃. In another embodiment, the preparation comprises about 0.5 mg/mL to about 3.0 mg/mL mg mannitol. In a specific embodiment, the preparation comprises about 0.625 mg/mL mannitol. In another specific embodiment the preparation comprises 1.25 mg/mL mannitol. In another specific embodiment, the preparation comprises about 100 mg/mL NaHCO₃, about 0.625 mg/mL mannitol and about 0.1 mg/mL EO9 in a solution comprising EDTA, PG and water.

One embodiment according to the present invention includes a pharmaceutical preparation comprising EO9, NaHCO₃ and mannitol in a solution comprising PG, EDTA and water wherein the PG is present in the solution in a percentage range selected from the group consisting of about 6% to about 14% vol/vol; about 16% to about 24% vol/vol, and about 26% to about 34% vol/vol. In another embodiment, the PG is present in the solution in a percentage selected from the group consisting of about 10% vol/vol, about 20% vol/vol, and about 30% vol/vol. In another embodiment, the preparation comprises a solution with about a 347 μM EO9 concentration and about a 10% vol/vol PG concentration. In yet another embodiment, the preparation comprises a solution with about a 347 μM EO9 concentration and about a 20% vol/vol PG concentration. In a further embodiment, the preparation comprises a solution with about a 347 μM EO9 concentration and about a 30% vol/vol PG concentration. These described embodiments of the present invention can comprise about 10 mg/mL to about 120 mg/mL NaHCO₃ and in one particular embodiment will comprise about 100, about 100.25 or about 50.125 mg/mL NaHCO₃. These described embodiments of the present invention can also comprise about 0.5 mg/mL to about 3.0 mg/mL mannitol and in one particular embodiment will comprise about 0.625 or about 1.25 mg/mL mannitol.

One embodiment of the present invention can include a pharmaceutical preparation wherein the preparation comprises a solution with about a 347 μM EO9 concentration, about a 10% vol/vol PG concentration, about 100.25 mg/ML NaHCO₃ and about 0.625 mg/mL mannitol. Another embodiment can include a pharmaceutical preparation wherein the preparation comprises a solution with about a 347 μM EO9 concentration, about a 30% vol/vol PG concentration, about 100.25 mg/mL NaHCO₃ and about 0.625 mg/mL mannitol.

### BRIEF DESCRIPTION OF THE FIGURES

FIG. 1 shows the immunohistochemical analysis of NQO1, P450R and Glut-1 in three patients with transitional cell carcinoma of the bladder.

FIG. 2 shows the apparatus used to study drug penetration through multicell layers.

FIG. 3 shows a schematic representation of drug solution preparations.

FIG. 4 shows a chromatogram of blank sample spiked with WV14 as an internal standard.

FIG. 5 shows chromatograms of EO9 standard in RPMI 1640 culture.

FIG. 6 shows chromatograms of EO9 standards in 0.1% DMSO (6A); 30% propylene glycol (PG; 6B); 20% PG (6C); and 10% PG (6D).

FIG. 7 shows calibration curves for EO9 in 0.1% DMSO and various PG (30%; 20%; 10%) concentrations.

FIG. 8 shows the penetration of EO9 in various PG concentrations through DLD-1 multicell layers.

FIG. 9 shows representative cross sections through stained DLD-1 multicell layers.

### DETAILED DESCRIPTION OF THE INVENTION

Quinone based bioreductive drugs are pro-drugs that generate cytotoxic species after enzymatic activation. The enzyme NAD(P)H:quinone oxidoreductase-1 (NQO1; also called DT-diaphorase (DTD)), a two electron reductase enzyme, plays a prominent role in the activation of quinone based bioreductive drugs under aerobic conditions. Quinone based bioreductive drugs are also cytotoxic under hypoxic conditions including cells with low NQO1 activity. One electron reducing enzymes such as Cytochrome P450 reductase may play a more prominent role in the activation of quinine based bioreductive drugs under hypoxic conditions. Based on the foregoing, the levels of these reductases and hypoxic conditions can indicate the appropriateness of different cancer therapies including the appropriateness of using various quinone based bioreductive drugs. The present invention thus evaluated levels of the described reductases and hypoxic condition in various grade and stage TCC.

Improvements in the treatment of bladder cancer can also occur based on providing pharmaceutical preparations comprising quinone based bioreductive drugs with varying penetration profiles. For example, pharmaceutical preparations with lower penetration profiles would be beneficial to use when treating superficial bladder cancers because the drug would remain nearer the surface of the bladder where treatment is most needed. Conversely, pharmaceutical preparations with higher penetration profiles would be beneficial when treating more muscle invasive bladder cancers because the drug would penetrate to deeper layers of the bladder where treatment is most needed in those cases. Taken together, the various aspects of the present invention provide important advancements in the treatment of bladder cancer by allowing the tailoring of cancer treatments to the particular characteristics of an individual's disease profile.

Apaziquone (prop. INN, USAN), also known as EO9 or NSC-382459 (3-hydroxymethyl-5-aziridinyl-1-methyl-2-(1H-indole-4,7-dione)-propenol with the structural formula:

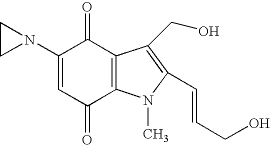

is a fully synthetic bioreductive alkylating indoloquinone. The basic mechanism of activation of EO9 is believed to be similar to that of other indoloquinones, involving reduction by cellular enzymes that transfer one or two electrons, forming semiquinone and hydroquinone, respectively. Oxidation of the semiquinone under aerobic conditions results in a redox cycle that can cause cell death by forming reactive oxygen species (ROS), resulting in DNA strand breaks. The semiquinone/hydroquinone can, particularly under hypoxic conditions, alkylate and crosslink DNA and other macromolecules, causing cell death. EO9 is one non-limiting example of a quinone based bioreductive drug that is appropriate for use with the present invention.

### Example 1

### I. Materials and Methods

A. Human Tissues

Formalin-fixed, paraffin-embedded specimens of human bladder transitional cell carcinomas (n=52) were used for this study after first obtaining consent from the local research and ethics committee (LREC) according to Medical Research Council regulations. All patient details were anonymised to ensure confidentiality and all experiments were performed in accordance with guidelines laid down by the LREC. The tumors used for the study were representative of all grades (11 Grade 1; 26 Grade 2; 15 Grade 3) of both superficial (19 pTa; 19 pT1) and muscle-invasive (14>pT2) stages of human bladder TCC. All tumor blocks were used for construction of tissue microarrays (TMAs) and subsequent immunohistochemical analysis.

B. Tissue Microarray Construction

Tissue microarray constructions (TMAs) were constructed from the paraffin embedded blocks to represent the various grades (G1-G3) and the various stages (pTa, pT1, ≧pT2) of human bladder TCC. Tissue microarray construction (TMA) was achieved using a Beecher Instruments microarrayer (Silver Spring, Md., USA) using a modified method of Bubendorf et al. (11) which is incorporated by reference herein. Briefly, sections of each paraffin embedded donor block were stained using hematoxylin and eosin (H&E), examined by microscopy and an area containing tissue of interest marked on the wax block. Cylindrical cores (600 μM) were punch-biopsied from these representative areas and transferred into a recipient block. Tissue sampling used four cores from each tumor block to provide representative data on each parent block. A total of 108 core samples representing 26 patients were included per TMA block and two TMA blocks were constructed. Sections, 5 μM thick, were cut from the recipient TMA blocks and mounted on glass slides using a tape transfer system (Instrumedics, USA). H&E staining for verification of histology and sample integrity was performed on the first and every subsequent tenth section cut from each microarray block. TMA slides were then subject to immunohistochemical analyses.

C. Antibodies

Antibodies used included a mouse monoclonal antibody against NQO1 (provided by Drs. Siegel and Ross, University of Colorado Health Sciences Center, Denver, USA), a goat polyclonal antibody specific for P450R (Santa Cruz Biotechnology, USA), a mouse monoclonal antibody against Ki67 (BD Biosciences, UK) and a rabbit polyclonal antibody specific for glucose transporter-1 (GLUT-1; Dako, UK).

D. Immunohistochemistry

Immunolocalisation of NQO1, P450R, GLUT-1 and Ki67 was assessed by immunohistochemistry, as previously described (9, 10, 12, 13) and understood by those of ordinary skill in the art. Briefly, following antigen retrieval and blocking of non-specific immunoglobulin binding, TMAs were incubated with the appropriate primary antibody: incubated for about 60 minutes with the anti-NQO1 antibody diluted in 1:1 TBSTM (10 mM Tris-HCl, 150 mM NaCl, 0.2% Tween 20, 5% non-fat dry milk powder); incubated for about 90 minutes for P450R diluted 1:100 in PBS; incubated for about 90 minutes with the anti-Glut-1 antibody diluted 1:25 in PBS; or incubated overnight at 4° C. with the anti-Ki67 antibody diluted 1:100 in PBS. Controls were performed using normal IgG instead of primary antibody. Immunolocalisation was achieved using the appropriate biotinylated secondary antibody (diluted 1:200; Vector Labs., USA), followed by signal amplification using a Vectastain ABC kit (Vector Labs., USA) and visualization with 3,3′-diaminobenzidine (DAB) (Vector Labs., USA). Sections were then counterstained with Harris' hematoxylin, dehydrated, cleared and mounted in DPX mountant (Sigma, UK).

E. Semiquantitative Analysis of Immunohistochemical Staining

Positive immunostaining was scored semi-quantitatively by three independent observers. Both NQO1 and P450R were localised cytoplasmically within the tumor. A score for the epithelial compartment of each tumor core based on intensity and distribution of stain was assigned from 0 (no staining) to 4 (maximal staining intensity). An average scoring intensity was calculated for each core and each tumor of the TMA from the results of the independent observers. The results were compared for any relationships and correlations to clinicopathological parameters.

The level of Glut-1 positivity in each TMA core was analysed and assigned a score from 0 to 4 representative of the approximate percentage of tumor cells demonstrating membrane staining (0=no staining; 1=0-5% positive; 2=5-15% positive; 3=15-30% positive; 4=>30% positive). An average scoring intensity was calculated for each core and each tumor of the TMA from the results of the independent observers. The results were compared for any relationships and correlations to clinicopathological parameters.

The percentage Ki67 positive nuclei in the tumor cells was calculated using 40× magnification for each core and tumor, as reported by Santos et al. (13, 14) which is incorporated by reference herein. A total of 200 cells per core and 800 cells per tumor were counted and the percentage positivity calculated. The scoring was performed independently by two observers. The results were compared for any relationships and correlations to clinicopathological parameters.

F. Statistical Analysis

The expression of NQO1 and P450R were compared with the following clinicopathological parameters: tumor stage, tumor grade, tumor hypoxia (Glut-1 expression) and proliferation. Statistical analysis was undertaken using the SPSS software package, version 11.0 (SPSS Inc., Chicago, Ill.). In the immunohistochemical study, because expression is not normally distributed, the average expression values for each category were reported as medians with interquartile ranges. Differences between independent variables were determined by the Mann-Whitney U test. Values of P less than 0.05 in two-tailed analyses were considered significant.

### II. Results

A. Relationship Between NQO1 Protein Levels, Tumor Stage and Grade

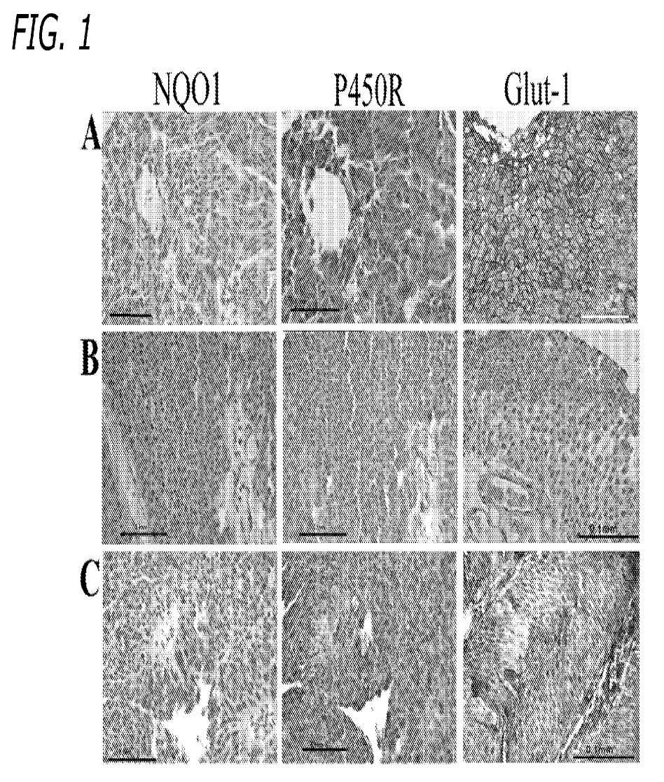

NQO1 was localised cytoplasmically in the epithelia of bladder tumors of all pathological grade and stage and expression of NQO1 varied between tumors (FIG. 1, Table 1). In many cases a heterogenous expression pattern of NQO1 was observed within the same tumor, with areas of high and low NQO1 expression within the same sample (data not shown). NQO1 was expressed in tumors of all pathological stage (pTa, pT1, >pT2) although expression levels of NQO1 varied between the various stages (Table 1). A significant difference in NQO1 expression was observed between superficial tumors (pTa+pT1) and muscle invasive tumors (≧pT2), with expression being significantly lower in muscle invasive tumors (P=0.02). The inverse relationship of NQO1 expression to tumor invasive potential is further reinforced by the significant difference in expression observed between non-invasive (pTa) and invasive (pT1+≧pT2) tumors (P=0.03). All pathological grades of TCC expressed NQO1 (Table 1). Expression of NQO1 was significantly higher in grade 2 tumors compared to either grade 1 or grade 3 (Table 1). No significant difference was observed between highly differentiated (grade 1) and poorly differentiated (grade 3) tumors (Table 1).

B. Relationship Between P450R Protein Expression and Tumor Stage and Grade

All tumors examined expressed detectable levels of P450R localised cytoplasmically. In contrast to NQO1, P450R expression was generally uniform within tumors. Representative immunostaining is depicted in FIG. 1. P450R was expressed in all stages of TCC (Table 1). Levels of P450R were significantly higher in muscle invasive tumors (>pT2) compared to superficial (pTa+pT1) tumors (P<0.01). In contrast to NQO1, expression of P450R shows a positive relationship to increasing tumor stage but is not associated with the invasive potential of the tumor, as is evident from the lack of significant difference observed between invasive (pT1+>pT2) and non-invasive (pTa) tumors (Table 1). All pathological grades of TCC expressed P450R (Table 1). A positive correlation was observed between P450R levels and increasing tumor grade (Table 1).

C. Relationship Between Glut-1 and Tumor Stage and Grade

The expression of Glut-1 protein was heterogenous both within individual tumor specimens and between individual patient samples. Representative immunostaining and its relationship with tumor stage and grade are presented in FIG. 1 and Table 1 respectively. Glut-1 protein was expressed in all stages and grades examined although levels of Glut-1 were significantly higher in >pT2 tumors (relative to pTa tumors, P=0.05) and Grade 3 tumors (relative to both Grade 1 [P=0.03] and Grade 2 [P<0.01] tumors). In addition, statistically significant differences (P=0.02) exist between non-invasive (pTa) and invasive (pT1+≧pT2) tumors suggesting that invasive disease is associated with higher Glut-1 protein expression and consequently higher levels of hypoxia.

D. Relationship Between Ki67, Tumor Stage, Tumor Grade and Enzymology

Expression levels of Ki67 antigen were used as an indicator of tumor proliferative index (Table 1). As expected, a significant correlation was observed between increasing tumor grade (decreasing differentiation) and proliferation index (P<0.01). No relationship was observed between tumor proliferation and tumor invasive potential (pTa versus pT1+≧pT2). In contrast, tumor proliferation was significantly higher in muscle invasive tumors (>pT2) relative to superficial tumors (pTa+pT1 [P<0.01]) probably as a result of the relationship between muscle invasion and higher tumor grade. Interestingly, a significant relationship was observed between tumor proliferative index and both Glut-1 expression (P=0.01) and P450R expression (P<0.01), but not NQO1 expression.

The results of this study demonstrate that the protein expression of key enzymes involved in the bioreductive activation of quinone based compounds and the presence of hypoxia as determined by Glut-1 protein levels changes with stage and grade of bladder TCC. The most striking observation is the fact that NQO1 protein expression decreases significantly with increasing tumor stage (Table 1). With regards to tumor grade, there is also evidence that G3 tumors have lower levels of NQO1 than G2 (but not G1) tumors. These findings are in agreement with previously published studies where an inverse relationship between NQO1 mRNA expression and increasing tumor stage (15) was reported. Similarly for Glut-1, increased protein expression with tumor grade (P=0.03 and <0.01 when G1 and G2 was compared with G3 tumors respectively) and tumor stage (P=0.05 when pTa tumors are compared to ≧pT2 tumors) is consistent with previous reports (16). In contrast to previously published reports demonstrating higher levels of P450R mRNA in superficial compared to muscle-invasive TCC (15), P450R protein levels were significantly higher in muscle-invasive (≧pT2 compared to pTa+pT1) disease in this study (P<0.01). In addition, P450R protein expression shows a positive correlation with increasing tumor grade (decreasing differentiation) (Table 1). Interestingly, P450R expression also demonstrated a strong positive correlation to proliferation index (P<0.01), probably as a consequence of a strong relationship between P450R, Ki67 and increasing tumor grade (decreasing differentiation). Nevertheless, this should be borne in mind when evaluating bioreductive therapies involving P450R since high proliferative index has been shown to relate to poor prognosis in bladder cancer (17, 18). In summary, analysis of protein expression by immunohistochemistry suggests that hypoxia, as demonstrated by Glut-1 expression, relates to increasing tumor stage, grade and tumor invasion. With reference to tumor enzymology, this study suggests NQO1 levels significantly decrease as a function of increasing tumor stage (and invasive potential) whereas P450R levels increase with tumor grade and invasive potential.

These findings have significant implications for potential therapeutic strategies using quinone based bioreductive drugs in the treatment of bladder TCC. There is extensive evidence in preclinical models indicating that the response of cells to MMC, EO9 and RH1 is dependent not only on NQO1 levels but also on the level of tumor hypoxia. With regards to MMC, the role of NQO1 in determining cellular response under aerobic conditions is controversial but under hypoxic conditions, significant potentiation of activity is seen only in cells that have low or no NQO1 activity (19). In the case of EO9 and RH1, similar results have been obtained under hypoxic conditions with marked potentiation of activity observed only in cells with low NQO1 (20, 21). Under aerobic conditions however, there is a good correlation between NQO1 activity and chemosensitivity suggesting that in the presence of oxygen, NQO1 plays a prominent role in activating EO9 and RH1 (22, 23). The mechanistic basis to explain these observations is not clear (24) but under hypoxic conditions, one electron reductases such as P450R assume a more influential role in the bioreductive activation process (25). Based on these findings, compounds such as EO9 and RH1 would target the aerobic fraction of NQO1 rich tumors (and so would MMC but to a lesser extent) or the hypoxic fraction of NQO1 deficient tumors assuming that one electron reductases such as P450R are present. In the case of NQO1 rich tumors therefore the use of compounds such as EO9 and RH1 as single agents targeting the aerobic fraction would be appropriate. For NQO1 deficient tumors with a significant hypoxic fraction, these agents should be used in combination with radiotherapy or other chemotherapeutic agents that target the aerobic fraction. The results of this study suggest that this latter strategy may be effective in the case of more advanced TCC of the bladder (i.e. ≧pT2) or more aggressive disease (i.e. Grade 3 tumors) as these typically have low NQO1 protein expression (and possibly greater P450R expression) and contain significant areas of hypoxia. In this specific context, it is of interest to note that encouraging results have been obtained in muscle invasive bladder cancer using chemoradiotherapy (Mitomycin C plus 5 Fluorouracil in combination with radical radiotherapy) although analysis of NQO1 and hypoxia markers was not incorporated into the design of this study (26). In the broader context, the demonstration in this and other studies that both superficial and muscle invasive bladder TCC have significant regions of hypoxia suggests that these tumors are attractive candidates for evaluating other bioreductive drugs or hypoxia mediated therapies.

In conclusion, the results of this study have demonstrated that the protein expression of key enzymes involved in the bioreductive activation of quinone based compounds and the presence of hypoxia changes as a function of tumor stage and grade in TCC of the bladder. These results suggest that these tumors (i.e. ≧pT2 and G3 tumors) would be good candidates for chemo-radiotherapy regimens using quinones (e.g. MMC, EO9 and RH1) to target the hypoxic fraction in combination with radiation or other chemotherapeutics to target the aerobic fraction of cells. Based on these rationales, and referring back to FIG. 1, case A (pT₂ G3) demonstrates low NQO1, high P450R and High Glut-1 levels and therefore would be a good candidate for chemoradiotherapy using quinones. Case B (pTa G1) has high NQO1, low P450R and moderate Glut-1 and as such should respond well to quinone based chemotherapy. Case C (pT₁ G2) which has moderate NQO1, moderate P450R and moderate Glut-1 would also be predicted to respond well to quinone based chemotherapy. Profiling of individual patients tumors for these markers remains important, particularly in view of the marked interpatient heterogeneity (particularly with NQO1) that exists.

As used herein, when using enzyme levels to determine an appropriate treatment for a patient, “high” versus “low” levels of the enzyme can be ascertained by comparing levels of the enzyme of interest from the relevant tumor to other tumors from the same patient, to tumors from another patient and/or to standard tumor cell lines or other available reference points known to those of ordinary skill in the art. Thus, “high” and “low” levels can be determined by a treating physician or other laboratory, research or treatment personnel involved in measuring and/or quantitating a particular patient's tumor enzyme levels.

### Example 2

### I. Materials and Methods

A. Apparatus and General Assay Principle

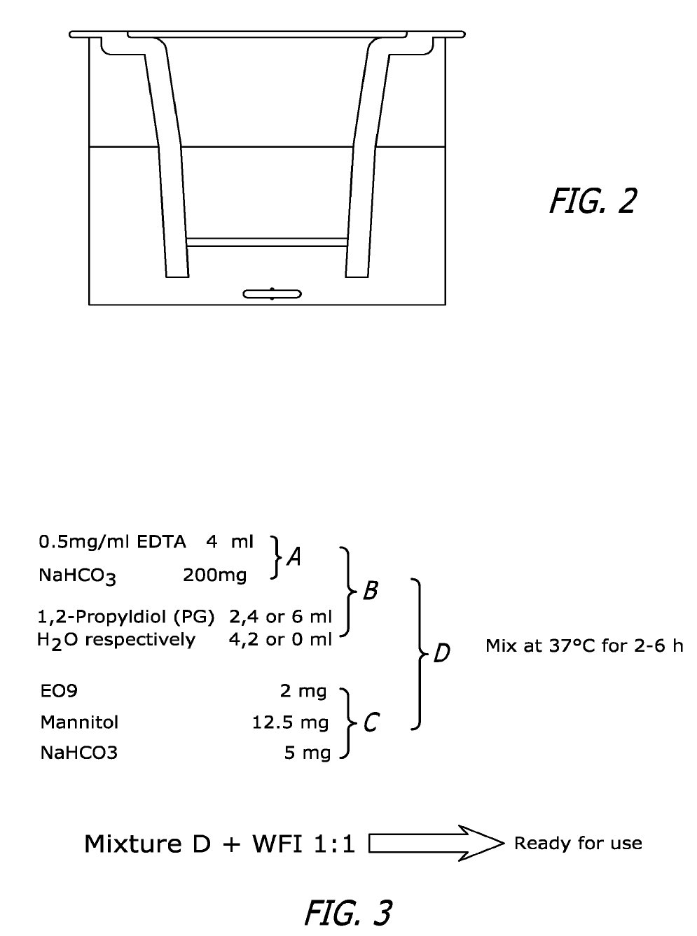

As shown in FIG. 2, the apparatus used in the described experiment comprised a transwell insert (Costar) inserted into one well of a 24 well culture plate. The insert had a collagen coated membrane at its base and thus formed both a barrier between the top and bottom chamber as well as a surface upon which cells could attach and grow. The cell line used in this study was DLD-1 human colon adenocarcinoma cells which was selected because of its ability to form tight junctions between cells thereby forming a continuous ‘barrier’ across which the drug must cross. To assess drug penetration, drugs were added to the top chamber and the concentration of drug in the bottom chamber was determined over a range of time intervals.

B. Cell Culture Conditions

DLD-1 cells were routinely maintained in RPMI 1640 medium supplemented with 10% fetal calf serum, sodium pyruvate (1 mM), L-glutamine (2 mM), penicillin/streptomycin (501 U/ml, 50 μg/ml) and buffered with HEPES (25 mM). DLD-1 cells (2.5×10⁵ in 200 μl of medium) were added to the top chamber and allowed to settle and attach to the membrane for approximately 3 hours at 37° C. in a CO₂ enriched (5%) atmosphere. Once cells attached, the transwell was inserted into one well of a 24 well plate and 600 μl media was added to the bottom chamber. The apparatus was then incubated at 37° C. for 4 days with daily media changes to both the upper and lower chamber. Based upon previous studies, the thickness of the multicell layer after 4 days of culture is approximately 50 μm. For each assay, 3 transwells were removed for histological examination and accurate determination of thickness and integrity (see below for details).

C. Preparation of Drug Solutions

The following solutions were prepared as described below and summarized in FIG. 3.

1. Solution 1: EO9 (347 μM) in 0.1% DMSO

Solid EO9 was dissolved in 100% DMSO to make a stock solution of 347 mM. 10 μl of the stock solution were added into 10 ml of complete RPMI medium (phenol red free). In order to prevent a possible precipitation of EO9, the addition of EO9 stock solution into the medium was with a continuous shaking. The final concentration of EO9 was 347 μM which is equivalent to 4 mg/40 ml.

2. Solution 2: EO9 (347 μM) in 10% PG

Two hundred milligrams of sodium bicarbonate (NaHCO₃) were dissolved in 4 ml of EDTA solution (0.5 mg/mL, which was prepared fresh from 0.5 M stock solution, Sigma). The solution was then mixed with 6 ml PG solution (2 ml PG+4 ml H₂O) making a final volume of 10 ml containing 20% PG. This solution was added into 20 ml universal tube containing EO9 (2 mg), sodium bicarbonate (5 mg) and mannitol (12.5 mg). The solution was incubated at 37° C. with continuous shaking until the EO9 was completely dissolved (about 5-6 hours). Then, the solution was diluted 1:1 with water to yield 10% PG, solution.

3. Solution 3: EO9 (347 μM) in 20% PG

Two hundred milligrams of sodium bicarbonate (NaHCO₃) were dissolved in 4 ml of EDTA solution (0.5 mg/mL, which was prepared fresh from 0.5 M stock solution, Sigma). The solution was then mixed with 6 ml PG solution (4 ml PG+2 ml H₂O) making a final volume of 10 ml containing 40% PG. This solution was added into 20 ml universal tube containing EO9 (2 mg), sodium bicarbonate (5 mg) and mannitol (12.5 mg). The solution was incubated at 37° C. with continuous shaking until the EO9 was completely dissolved (about 3-4 hours). Then, the solution was diluted 1:1 with water to yield 20% PG, solution.

4. Solution 4: EO9 (347 μM) in 30% PG

Two hundred milligrams of sodium bicarbonate (NaHCO₃) were dissolved in 4 ml of EDTA solution (0.5 mg/mL, which was prepared fresh from 0.5 M stock solution, Sigma). The solution was then mixed with 6 ml PG (6 ml PG+0 ml H₂O) making a final volume of 10 ml containing 60% PG. This solution was added into 20 ml universal tube containing EO9 (2 mg), sodium bicarbonate (5 mg) and mannitol (12.5 mg). The solution was incubated at 37° C. with continuous shaking until the EO9 was completely dissolved (about 2 hours). Then, the solution was diluted 1:1 with water to yield 30% PG, solution.

D. Drug Administration

Throughout all procedures, the media used was as described above except for the fact that phenol red free media was used (phenol red elutes very close to EO9 on the chromatograms). EO9 was added to the top chamber at t=0 in a volume of 100 μl and the bottom chamber contained 600 μl of media (constantly stirred). Following a 10 minute incubation at 37° C., the transwell was removed and placed into a new well of the 24 well plate containing 600 μl of fresh media. The drug solution in the top chamber was removed and replaced with 100 μl of fresh drug solution (i.e., the concentration in the top chamber was maintained at a constant concentration). This whole procedure was repeated at 10 minute intervals over a total time period of 1 hour.

E. Extraction Procedures

EO9 was immediately extracted using Isolute C18 SPE cartridges. Cartridges were primed with 1 ml methanol followed by washing in 1 ml deionised water prior to sample addition (500 μl). Following a further washing in 1 ml deionised water, EO9 was eluted in 300 μl methanol. Samples were dried under vacuum (at room temperature in a rotary evaporator) and either stored at −20° C. until required for analysis or reconstituted in mobile phase (see below) for immediate analysis.

F. HPLC Analysis

Chromatographic analysis of EO9 was carried out as described by Phillips et al. (British Journal of Cancer. 65(3):359-64, 1992) which is incorporated by reference herein. Briefly, a Hichrom RPB column (25 cm×4.6 mm id, Hichrom Ltd, UK) was used for the separation. A Waters 996 Photodiode Array Detector (λ₁=280 nm,) with Masslynx 3.4 software (Micromass Ltd) was used for spectral analysis of the peaks of interest. The mobile phase consisted of 1 M phosphate buffer (1%), methanol (42%) and HPLC grade water (57%). The flow rate was set at 1.2 ml min⁻¹ using a Waters Alliance 2690 (Milford, Mass., USA) quaternary pump chromatography system, which also incorporates the autosampler. The detection limit was 10 ng/ml (34.7 nM).

G. Histology

For each experiment, 3 transwell inserts were collected; 1 control and 2 at the end of the experiment. Each transwell was fixed in 10% formalin for one hour prior to transfer to 70% ethanol and storage overnight. Using a clean scalpel, the membranes were carefully detached from the plastic insert and processed for embedding in paraffin wax using standard procedures known to those of ordinary skill in the art. Specimens were sectioned (5 μm) using a Leitz rotary microtone, mounted onto protein coated glass slides and stained using haemotoxylin and eosin also using standard procedures known to those of ordinary skill in the art. The thickness of the multicell layer was measured using an eyepiece graticule that had been calibrated using a stage micrometer. Five measurements were obtained for each section and 3 sections per sample were measured.

### II. Results

A. Representative Chromatograms

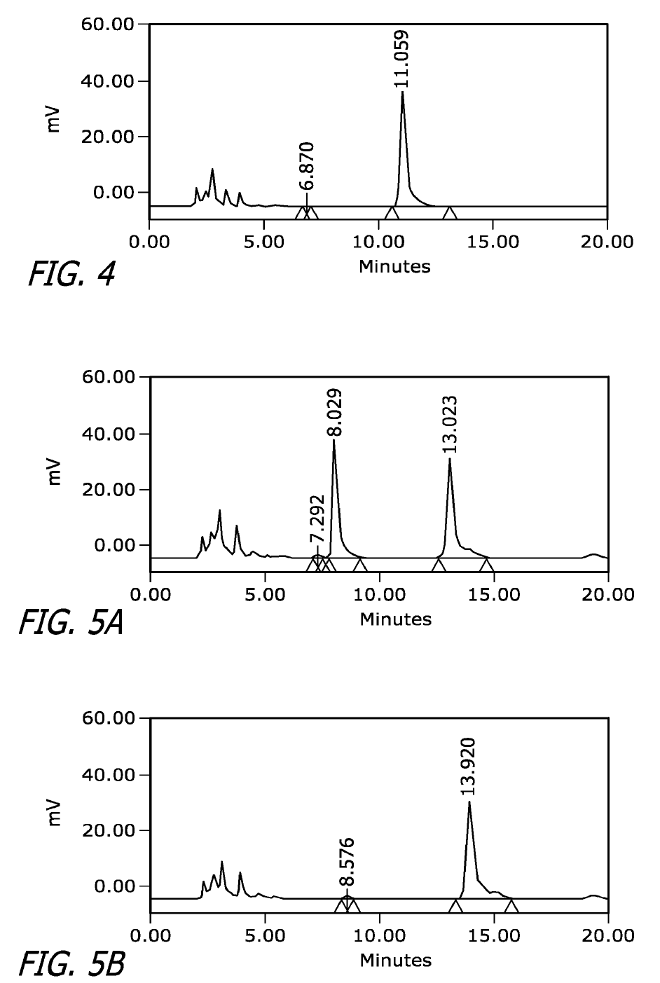

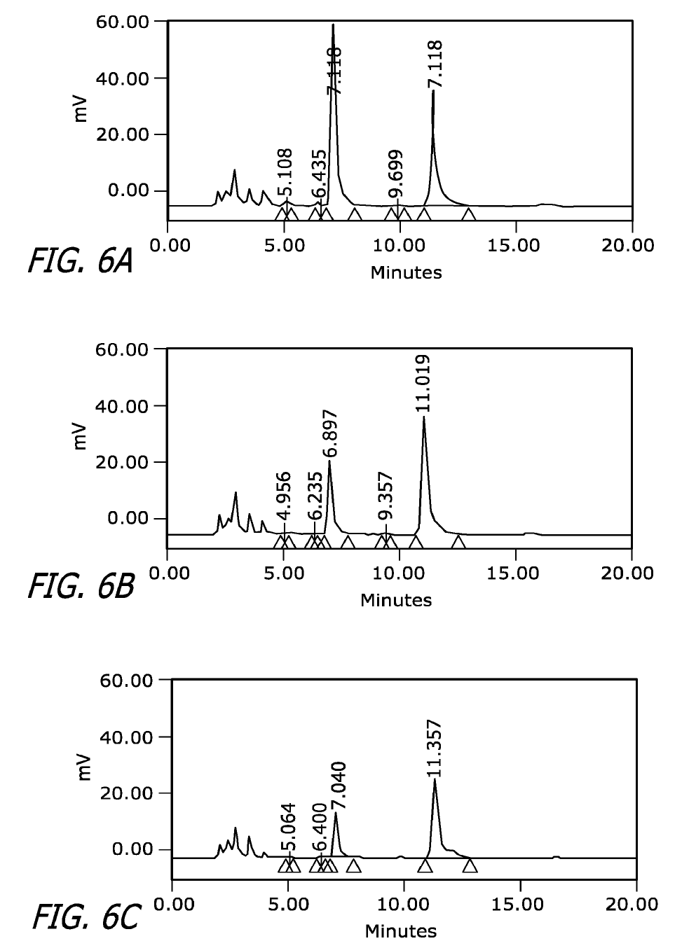

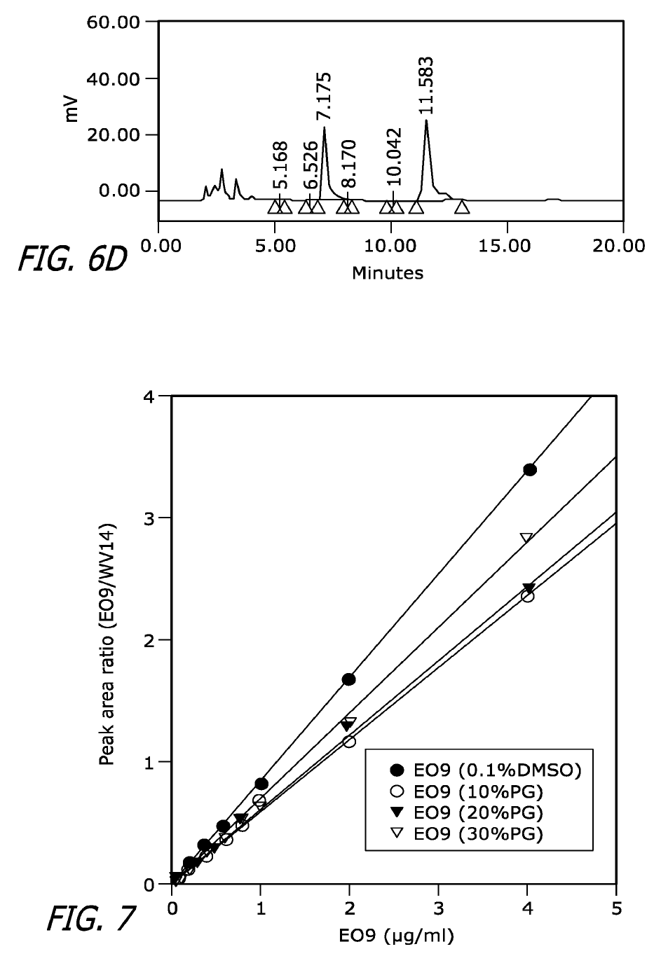

FIG. 4 shows a chromatogram of a blank sample spiked with WV14 internal standard (retention time=11.059 minutes). The peak at 6.870 minutes is a contaminating peak. FIG. 5 shows EO9 standards (1 μg/ml (FIG. 5A) and 20 ng/ml (FIG. 5B)) in RPMI 1640 culture medium. As shown in FIG. 5A, the EO9 and WV14 peaks elute at 8.029 minutes and 13.023 minutes respectively (the peak at 7.292 min is the contaminating peak described above). It should be noted that retention times can move due to temperature fluctuations in a laboratory but that relative retention times should remain constant. FIG. 5B indicates the limit of detection. FIG. 6 shows chromatograms of EO9 standards in 0.1% DMSO (FIG. 6A); 30% PG (FIG. 6B); 20% PG (FIG. 6C); and 10% PG (FIG. 6D).

B. Calibration Curves

Calibration curves were constructed for each EO9 preparation and the results are presented in FIG. 7. Calibration curves were reproducible and subtle differences in the slope of each calibration curve were observed as illustrated in FIG. 7. The reasons for the differences are unclear but may reflect slight differences in extraction efficiency between the different preparations. The extraction efficiencies for EO9 in 0.1% DMSO, 10% PG, 20% PG and 30% PG were 92.3%, 81.7%, 79.9% & 81.1% respectively. Because of this variation, calibration curves were generated for each experiment conducted. No obvious breakdown products were visible on any of the chromatograms.

C. Drug Penetration

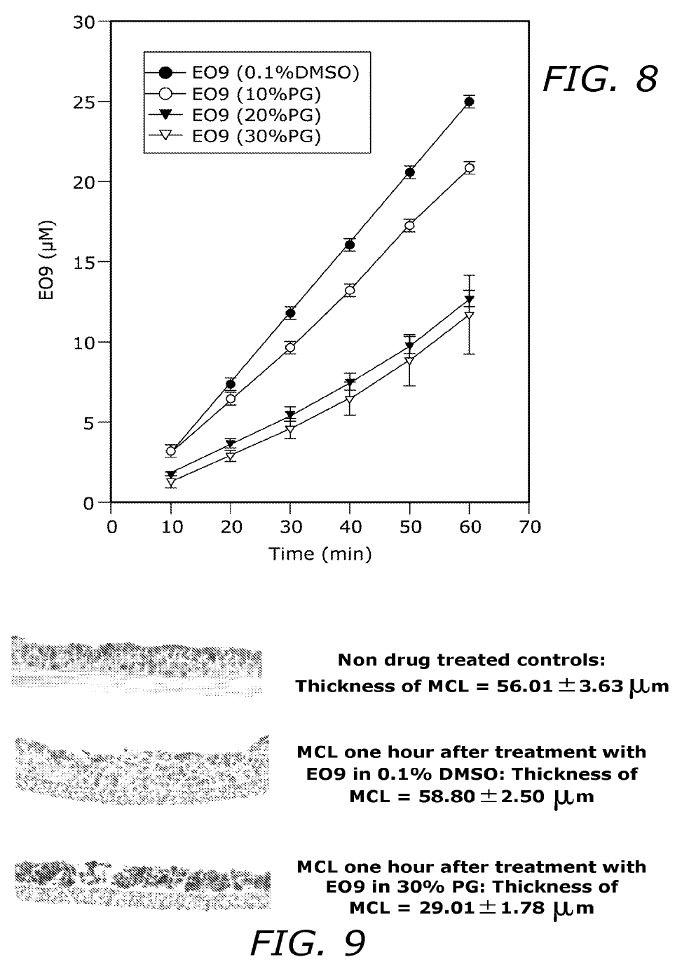

As can be seen in FIG. 8, as the concentration of PG increases, the multicell layer penetration rate of EO9 decreases. With regard to EO9 in 0.1% DMSO, the kinetics is linear which is as expected when the concentration in the top chamber is maintained at a more or less constant value. At the two highest concentrations of PG tested, it is worth noting that the kinetics are not quite linear—there is a progressive increase in rate as time increases. This effect probably reflects the changes in the thickness of the multicell layer induced by PG (see FIG. 9). No obvious metabolites or breakdown products were observed at any of the evaluated time points.

FIG. 9 shows the results of histological analyses undertaken to examine the penetration of EO9 through DLD-1 multicell layers. The thickness of non-drug treated sections was 56.01±3.63 μm. After one hour of treatment with EO9 in 0.1% DMSO, the thickness of the multicell layer was not significantly different from non-drug treated specimens (58.80±2.50 μm). Following treatment with EO9 in 30% PG however, the thickness of the multicell layer decreased significantly to 29.01±1.78 μm. There were also marked morphological changes in appearance within the layer, the most obvious of which was the appearance of ‘breaks’ or ‘channels’ in the layer itself. An observation made throughout experiments using EO9 in PG was that the upper chamber contained more fluid than expected. For example, after a 10 min incubation with EO9 in PG at 30%, 20% and 10%, the volume recovered from the top chamber was 106±3, 107±3 and 105±2 μl respectively (after a one hour exposure to EO9 in 0.1% DMSO, the volume recovered was 98±2 μl). It should be stressed that these volumes are only approximations (being based on what could be recovered using a Gilson pipette) but they do indicate that the volume of media in the upper chamber changes when EO9 dissolved in PG formulations (especially at 30% PG) is used. It is also noteworthy that the histological pictures show that cells are in close contact with the basement membrane in controls and EO9 (0.1% DMSO) treated specimens but for multicell layers treated with EO9 in 30% PG, there is a small but distinct gap between the multicell layer and the membrane itself.

TABLE 1
Protein expression of NQO1, P450R, GLUT-1 and Ki67 in human TCC of the bladder.

| | | | | | % |
|---|---|---|---|---|---|
| | Number | Median NQO1 | Median P450R | Median GLUT-1 | proliferation |
| | of | expression | expression | expression | (Ki67positive) |
| | Samples | (±interquartiles) | (±interquartiles) | (±interquartiles) | (±S.E.) |
| pTa | 19 | 2.50 (1.14-3.20) | 3.20 (2.58-3.83) | 2.00 (1.30-3.80) | 16.75 ± 2.8 |
| pT₁ | 19 | 1.88 (0.33-3.00) | 2.96 (2.33-3.67) | 3.38 (2.75-3.88) | 13.88 ± 2.2 |
| pT₂ | 14 | 0.17 (0.00-1.67) | 3.89 (3.75-3.92) | 3.88 (2.67-4.00) | 24.59 ± 4.43 |
| G1 | 11 | 1.00 (0.00-1.10) | 2.79 (2.17-2.92) | 2.38 (2.00-3.25) | 9.72 ± 2.64 |
| G2 | 26 | 2.72 (1.83-3.20) | 3.35 (2.75-3.83) | 2.83 (1.75-3.75) | 14.59 ± 1.72 |
| G3 | 15 | 0.33 (0.00-1.85) | 3.83 (3.31-3.92) | 4.00 (3.63-4.00) | 30.47 ± 3.71 |
| Non- | 19 | 2.50 (1.14-3.20) | 3.20 (2.58-3.83) | 2.00 (1.31-3.67) | 17.51 ± 2.83 |
| invasive^{a} | | | | | |
| Invasive^{b} | | | | | |
| Superficial^{c} | 33 | 1.67 (0.0-2.52) | 3.67 (2.92-3.89) | 3.50 (2.71-4.00) | 19.41 ± 2.86 |
| Muscle | 38 | 2.00 (1.08-3.17) | 3.10 (2.33-3.78) | 2.83 (1.83-3.83) | 15.69 ± 1.79 |
| Invasive^{d} | 14 | 0.17 (0.00-1.67) | 3.89 (3.75-3.92) | 3.88 (2.67-4.00) | 24.59 ± 4.43 |

Data for NQO1, P450R and GLUT-1 are presented as the median score (±interquartile range) of two observers.
Data for proliferation index are presented as mean score ± S.E of two observers.
Specimens were rated between 0 and 4 for NQO1, P450R and GLUT-1 and proliferation index was calculated as % Ki67 positivity as described in “Materials and Methods”.
The suffixes a, b, c and d denote pTa; (pT₁ + pT₂); (pTa + pT₁) and pT₂ tumour stages respectively.

What is claimed is:

1. A pharmaceutical preparation comprising EO9 in a solution with a propylene glycol (PG) concentration selected from the group consisting of about 30% vol/vol PG, about 20% vol/vol PG, and about 10% vol/vol PG.

2. A pharmaceutical preparation according to claim 1 wherein said preparation comprises a solution with about a 300 μM to about a 400 μM EO9 concentration.

3. A pharmaceutical preparation according to claim 1 wherein said preparation comprises a solution with about a 347 μM EO9 concentration.

4. A pharmaceutical preparation according to claim 1 wherein said preparation further comprises NaHCO₃, EDTA, mannitol and water.

5. A pharmaceutical preparation according to claim 4 wherein said preparation comprises from about 10 mg/mL to about 120 mg/mL NaHCO₃.

6. A pharmaceutical preparation according to claim 5 wherein said preparation comprises about 100 mg/m NaHCO₃.

7. A pharmaceutical preparation according to claim 5 wherein said preparation comprises about 50 mg/mL NaHCO₃.

8. A pharmaceutical preparation according to claim 4 wherein said preparation comprises about 0.5 mg/mL to about 3.0 mg/mL mannitol.

9. A pharmaceutical preparation according to claim 8 wherein said preparation comprises about 0.625 mg/mL mannitol.

10. A pharmaceutical preparation according to claim 8 wherein said preparation comprises about 1.25 mg/mL mannitol.

11. A pharmaceutical preparation according to claim 1 wherein said preparation comprises about 100 mg/mL NaHCO₃, about 0.625 mg/mL mannitol, and about 0.1 mg/mL EO9 in a solution comprising EDTA, PG and water.

12. A pharmaceutical preparation comprising EO9, NaHCO₃ and mannitol in a solution comprising PG, EDTA and water wherein said PG is present in said solution in a percentage range selected from the group consisting of about 6% to about 14% vol/vol; about 16% to about 24% vol/vol, and about 26% to about 34% vol/vol.

13. A pharmaceutical preparation according to claim 12 wherein said PG is present in said solution in a percentage selected from the group consisting of about 10% vol/vol, about 20% vol/vol, and about 30% vol/vol.

14. A pharmaceutical preparation according to claim 12 wherein said preparation comprises a solution with about a 347 μM EO9 concentration and about a 10% vol/vol PG concentration.

15. A pharmaceutical preparation according to claim 12 wherein said preparation comprises a solution with about a 347 μM EO9 concentration and about a 20% vol/vol PG concentration.

16. A pharmaceutical preparation according to claim 12 wherein said preparation comprises a solution with about a 347 μM EO9 concentration and about a 30% vol/vol PG concentration.

17. A pharmaceutical preparation according to claim 12 wherein said preparation comprises about 10 mg/mL to about 120 mg/mL NaHCO₃.

18. A pharmaceutical preparation according to claim 17 wherein said preparation comprises about 100 mg/mL NaHCO₃.

19. A pharmaceutical preparation according to claim 17 wherein said preparation comprises about 50 mg/mL NaHCO₃.

20. A pharmaceutical preparation according to claim 12 wherein said preparation comprises about 0.5 mg/mL to about 3.0 mg/mL mannitol.

21. A pharmaceutical preparation according to claim 20 wherein said preparation comprises about 0.625 mg/mL mannitol.

22. A pharmaceutical preparation according to claim 20 wherein said preparation comprises about 1.25 mg/mL mannitol.

23. A pharmaceutical preparation according to claim 12 wherein said preparation comprises a solution with about a 347 μM EO9 concentration, about a 10% vol/vol PG concentration, about 100.25 mg/mL NaHCO₃ and about 0.625 mg/mL mannitol.

24. A pharmaceutical preparation according to claim 12 wherein said preparation comprises a solution with about a 347 μM EO9 concentration, about a 30% vol/vol PG concentration, about 100.25 mg/mL NaHCO₃ and about 0.625 mg/mL mannitol.
